(19) United States
(12) Patent Application Publication
(10) Pub. No.: US 2008/0219407 A1
(43) Pub. Date: Sep. 11, 2008

(54) Particle therapy system

(76) Inventors: Werner Kaiser, Erlangen (DE); Tim Use, Nuremberg (DE)

Correspondence Address: BRINKS HOFER GILSON & LIONE, P.O. BOX 10395, CHICAGO, IL, 60610, US

(21) Appl. No.: 12/074,351

(22) Filed: Mar. 3, 2008

(30) Foreign Application Priority Data
Mar. 8, 2007 (DE) 10 2007 011 399.6

(51) Int. Cl. A61N 5/10

(52) U.S. Cl. 378/65

(57) ABSTRACT

A particle therapy system is disclosed, including a treatment chamber and a rotatable gantry with a radiation treatment unit, capable of emitting particles at a variety of angles. A movably supported floor is provided in the treatment chamber, capable of being rotated about the same axis as the gantry, and an imaging modality is disposed on a surface of the movably supported floor. The movably supported floor supports the weight of the imaging modality and is configured so that the imaging modality may be placed in a parked position when the gantry is being rotated. The imaging modality may be a C-arm X-ray device positionable by a robot.

This application claims the benefit of German patent application DE 10 2007 011 399.6, filed on Mar. 8, 2007, which is incorporated herein by reference.

### TECHNICAL FIELD

The present application relates to a particle therapy system having an imaging device disposable in the treatment chamber.

### BACKGROUND

In particle therapy, as used, for example, for the treatment of cancer, a particle beam is generated using protons or heavy ions in a suitable particle accelerator. The particle beam is guided in a radiation channel and emerges in a treatment chamber through an exit window of the radiation channel. In many cases, because of the complicated beam guidance mechanisms, only a stationary beam exit window is provided. A rotatable gantry with an exit window is provided in some systems. However, the gantry is very bulky in its construction. The gantry surrounds a cylindrical treatment chamber, into which a treatment table is moved. For the most precise possible treatment, the patient tissue that is to be irradiated must be positioned in the isocenter of the system (that is, the point struck by the beam when the gantry is rotated).

At the end of the radiation channel in a radiation treatment unit, also called a nozzle, at least one beam detector as well as passive beam elements are typically located immediately before the exit window. To enable irradiating the patient from below as well, the gantry is ideally rotatable by 360° about the patient. The radiation treatment unit must also be rotatable in the region below the patient and the floor of a treatment chamber with a gantry is typically movable.

A precise orientation of the patient relative to the particle beam is necessary so that the particle beam will strike the region of the patient that is to be treated as precisely as possible. To assure and monitor this precise orientation, an imaging device in the treatment chamber allows monitoring of the position of the patient or the region to be treated. An imaging device of this kind includes an X-ray source and an X-ray detector, with which X-ray images of the patient to be treated can be obtained. However, other imaging systems may be employed as well, such as a positron emission tomography scanner.

In the case of treatment chambers with a rotatably supported gantry, the disposition of the imaging device is a challenge, because of space problems and because of the many movable elements that can collide with the imaging device.

In German Patent Disclosure DE 10 2004 048 212 A1, for instance, a radiation therapy system with an imaging device is disclosed. One X-ray detector is disposed laterally on each side of a therapy beam exit, and one X-ray emitter is diametrically opposite each X-ray detector. Positioning the X-ray systems is done by rotating the entry gantry.

### SUMMARY AND DESCRIPTION

A particle therapy system with a treatment chamber with a rotatably supported gantry is disclosed, in which treatment chamber an imaging modality is disposed so as to enable the use of the imaging modality, substantially independently of the position of the gantry. The particle therapy system may include: a treatment chamber, a rotatable gantry having a radiation treatment unit, whereby a particle beam can be emitted from various angles in the treatment chamber, a movable floor in the treatment chamber; and, an imaging modality, disposed on the movable floor.

The movable floor is configured and disposed so that it can stably bear the imaging modality. In this way, it is possible for the imaging device to be disposed in the treatment chamber in a space-saving way. The gantry may be rotatably supported about an axis of rotation, and the gantry may surround the treatment chamber.

The imaging modality may be fixedly disposed on the movably supported floor and may remain in the treatment chamber during the performance of treatment. The imaging modality, which may include a C-arm X-ray device may be positioned in an appropriate point for imaging.

In an example, the movable floor has at least one floor segment, on which the imaging device is disposed. The floor segment remains inside the treatment chamber regardless of the orientation of the gantry. As such, the imaging device may be used for imaging regardless of the position of the gantry.

The floor segment may be a one-piece element, and the imaging device may be disposed on the floor segment. The floor segment is surrounded by the gantry itself. As a result, the imaging modality may be located inside the gantry, such that the imaging device is close to the patient at every position of the gantry.

In another aspect, the movable floor is movably supported on the gantry. The weight of the imaging modality or the weight of the floor may be supported by the gantry itself, or by way of the gantry. The movable floor may be supported on a side wall of the gantry, and the side wall of the gantry may extend beneath the movable floor, so that the bottom may be supported by the side wall.

The floor has a geometry that is adapted to the gantry so that motion of the gantry and floor can be accomplished without requiring complicated adaptation of the motions to one another.

In sill another aspect, the imaging modality may include a mounting device and an imaging unit. The imaging unit can be positioned in various positions by the mounting device and the position of the imaging unit in space may be varied. This makes it possible to dispose the imaging unit in various positions for imaging. The imaging unit may be, for instance, a C-arm, with an X-ray source or X-ray detector mounted on an end thereof.

In an aspect, the mounting device for the imaging unit may be a robot arm, which may have a plurality of joints. The imaging unit maybe positioned in a “parked” position so that the imaging unit is located in proximity to a back wall of the gantry. In this way, the imaging unit can be parked in a position in which it is substantially protected against collisions.

### BRIEF DESCRIPTION OF THE DRAWINGS

FIG. 1 shows one embodiment of a front view on a cylindrical treatment chamber of a particle therapy system having an imaging unit;

FIG. 2 shows a longitudinal section through the cylindrical treatment chamber of FIG. 1; and

FIG. 3 shows a schematic representation of a particle therapy system according to one embodiment.

### DESCRIPTION

Exemplary embodiments may be better understood with reference to the drawings, but these examples are not intended to be of a limiting nature. Like numbered elements in the same or different drawings perform equivalent functions. When a specific feature, structure, or characteristic is described in connection with an example, it will be understood that one skilled in the art may effect such feature, structure, or characteristic in connection with other examples, whether or not explicitly stated herein.

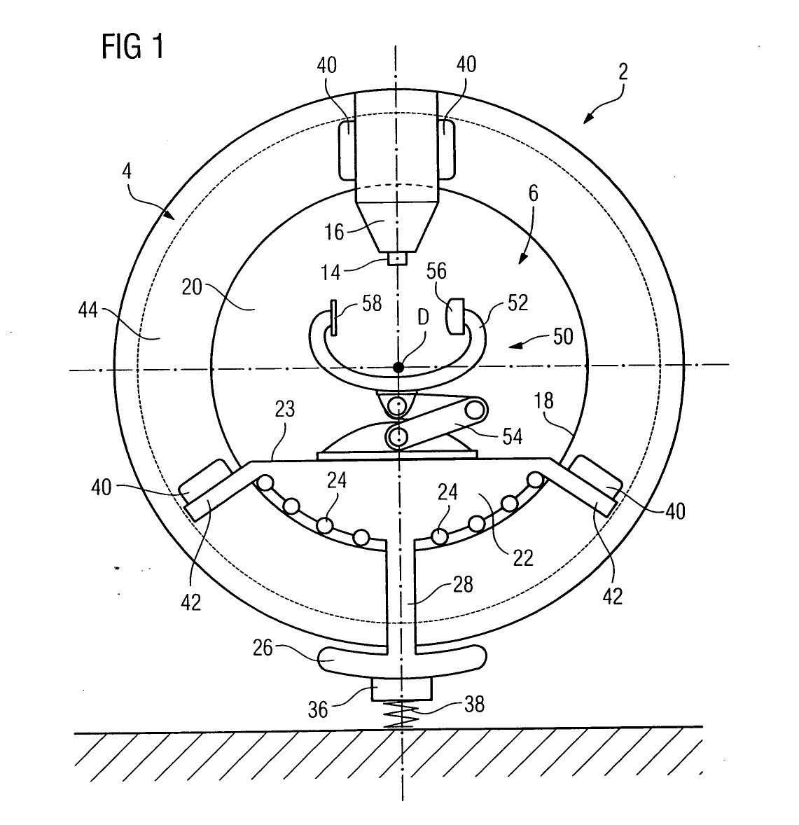

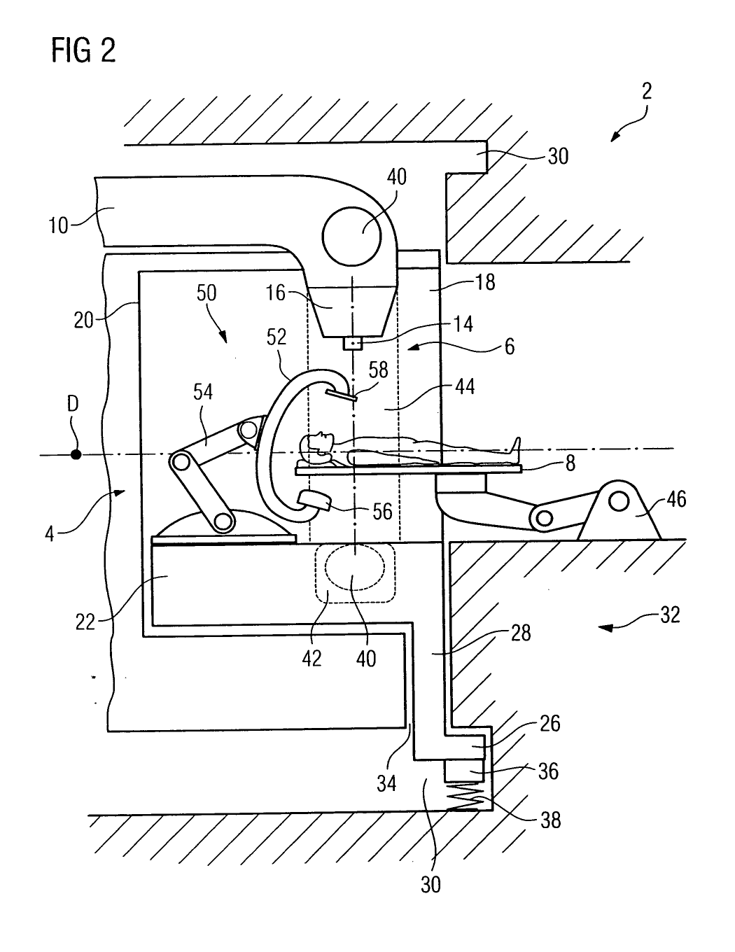

FIG. 1 and FIG. 2, show a treatment chamber 6 of a particle therapy system 2 with a gantry 4 that is rotatable about an axis of rotation D. The axis of rotation D, in the view of the particle therapy system in FIG. 1, extends perpendicular to the plane of the drawing. The gantry 4 surrounds the approximately cylindrical treatment chamber 6, in which a treatment table 8 can be positioned. The gantry 4 also surrounds a radiation channel 10, a partial view of which is shown. A particle beam (not shown), such as a heavy-ion or proton beam for treating a patient 12 lying on the treatment table 8, is guided in the radiation channel 10. The particle beam enters the treatment chamber 6 through an exit window 14 of a radiation treatment unit 16. The radiation treatment unit 16 protrudes from a rotatable, load-bearing side wall 18 of the treatment chamber 6. The back of the treatment chamber 6 may be bounded by a back wall 20, which may be a surface without load-bearing capability and which rotates with the gantry 4 about the axis of rotation D.

The treatment chamber 6 has a floor, which comprises a single, solid floor segment 22 that is supported by the side wall 18 of the gantry 4. In the exemplary embodiment shown, the floor segment 22 has assumed a neutral (horizontal) position so that a floor surface 23 may be walked on.

An imaging modality 50 may be disposed on and attached to floor surface 23. The imaging modality 50 may include an imaging unit 52, such as a C-arm, on the ends of which an X-ray emitter 56 and an X-ray detector 58 are disposed opposing each other. Using a mounting device 54, such as a robot arm with a plurality of joints, the imaging unit 52 can be positioned in space. The position of the patient 8 is monitored with the imaging unit 52 before or during the treatment. The floor surface may also be have a wall or a ceiling, and the imaging device 50 may be joined to those surfaces.

The imaging unit 52 can be positioned in a parked position, as shown in FIG. 1. In the parked position, the imaging unit 52 is located in proximity to the back wall 20 of the gantry 4. In this position, the imaging device 50 may not be a hindrance either to the positioning of the patient 8 or to irradiating the patient 8 with the particle beam. When imaging is desired, the imaging unit 52 may be moved out of the parked position and positioned at the desired position with respect to the patient 8. One possible position during operation of the imaging device 50 is shown in FIG. 2.

The load-bearing capacity of the floor segment 22 is selected to suit structural requirements, so that the floor segment 22 can support the imaging device 50 and any persons (not shown) operating the equipment.

The floor segment 22 may be supported on the side wall 18 using bearings 24, which may be roller bearings, so that the floor segment 22 and the side wall 18 are movable relative to one another. The gantry 4 may then rotate, while the floor segment 22 remains in a horizontal position, at least for some rotation angles. Alternatively, the floor segment 22, may be driven by an external force using, for example, the roller bearings 24, and can execute a motion about the axis of rotation D while the gantry 4 is stationary. The floor segment 22 may be rotated about the axis of rotation D, so that the relative positions of the floor segment 22 and the gantry 4 remains fixed, and the floor segment 22 and the gantry 4 may rotate together.

The floor segment 22 may have a cantilever 26, which is joined to the floor segment 22 via a strut 28 that extends perpendicular to the horizontal floor surface 23. A sliding block 30, for guiding the cantilever 26 and the strut 28 in moving the floor segment 22 about the axis of rotation D, extends around the treatment chamber 6 and may be a fixed floor 32 adjoining the treatment chamber. A shaft 34 may be formed between the gantry 4 and the fixed floor 32. A friction element 36 may be provided to engage the cantilever 26 and is elastically supported via a spring 28, so as to hold the floor segment 22 in the horizontal position.

The arrangement shown in FIGS. 1 and 2 permits a large number of irradiation angles while the movable floor segment remains stationary in the treatment chamber 6. For example, beginning at the position shown, where the radiation treatment unit 16 is perpendicular to the movable floor segment 22, a rotation of the radiation treatment unit 16 by at least 90° clockwise and counterclockwise about the axis D is possible, without the threat of a collision with the floor segment 22. Within this angular range, the floor segment 22 need not be moved out of the way. Not until a deflection of the radiation treatment unit 16 out of the position shown by an angle of rotation that is greater than at least ±90° is a displacement of the floor segment 22 effected about the axis of rotation D. For that purpose, the floor segment 22 may have an drive mechanism, so that, triggered by a control unit, the floor segment 22 is moved about the axis of rotation D before contact occurs between the floor segment 22 and the radiation treatment unit 16. However, in the present example, a separate drive mechanism or triggering of the floor segment 22 may not be provided. Movement of the floor segment 22 takes place as the radiation treatment unit 16 rotates and pushes the floor segment 22.

When the floor segment 22 is moved about the axis of rotation D, then the imaging device 50 has a corresponding movement. To avoid the risk of possible collisions of the imaging device 50 with other elements of the system or the patient, the imaging device 50 can be moved into the parked position during this period of motion. When the floor segment 22 has been displaced about the axis of rotation D and the gantry 4 comes to a stop, an image may be again made with the imaging device 50. For that purpose, the angle of the floor segment 22 relative to the neutral position may be determined using, for example, an angle encoder, an inclinometer, or the like.

To avoid a hard impact between the radiation treatment unit 16 and the floor segment 22, resilient buffer elements 40 may be disposed on both the radiation treatment unit 16 and the floor segment 22, in the region of a contact point between the floor segment 22 and the radiation treatment unit 16. To achieve a large area of contact between the floor segment 22 and the radiation treatment unit 16, the floor segment 22 may have two inclined vanes 42, one on each side, which extend outside the treatment chamber 6 and support the buffer elements 40 of the floor segment 22. Due to the inclined position of the vanes 42 with respect to the floor surface 23, a favorable angle is established in particular, so that the buffer elements 40 of the floor segment 22 and of the radiation treatment unit 16 may contact one another over a large area once the radiation treatment unit 16, in its rotation, reaches the floor segment 22. To permit the vanes 42 to be disposed outside the treatment chamber, an indentation 44 may be provided in the side wall 18; the indentation is represented in the drawings by a dashed line.

In this example, the floor segment 22 may be of a lightweight metal construction, so that the drive mechanism of the gantry 4, upon displacement of the floor segment 22, may be stressed as little as possible by the radiation treatment unit 16. Upon a deflection of the radiation treatment unit 16 from a vertical position shown in the drawings by a deflection angle of greater than 180°, the components of the force of gravity of both the floor segment 22 and the radiation treatment unit 16 are added together and act in the direction back toward the neutral position. This represents a stress on the drive mechanism of the gantry 4. So that the drive mechanism may not be stressed unnecessarily, the radiation treatment unit 16 is moved out of its vertical position by only up to 180° clockwise or counterclockwise, however, the entire angular range of about 360° is still covered.

The system 2 is operated by moving the treatment table 8 into the treatment chamber 6 using a robot arm 46. The treatment table 8 may not contact the floor segment 22 in this process. The robot arm 46 may be a multi-axial industrial robot arm with a multi-part mechanism and may be mounted on the stationary floor 32. With the aid of the robot arm 46, the treatment table 8 may be moved translationally in both the horizontal and the vertical directions. Rotation of the robot arm 46 about a plurality of axes is possible, so that the motion of the treatment table 8 may be characterized by three degrees of freedom translationally and three degrees of freedom rotationally. As a result of the translational and rotary motion of the treatment table 8, the location and the distance of the patient 12 relative to the radiation treatment unit 16 may be adjusted. During positioning of the treatment table 8 in the treatment chamber 6, the treatment table 8 may remain in a horizontal position, so that the patient 12 is lying down in a stable position.

The treatment chamber 6, of the particle therapy system 2, thus permits irradiation of the patient 12 from a variety of angular positions of rotation about the axis of rotation D. Moving the radiation treatment unit 16 beneath the treatment table 8 is possible, as the floor segment 22, may be moved out of the way of the motion of the radiation treatment unit 16. In this example, additional drive mechanisms or separate triggering of the floor segment 22 may not be required. The floor segment 22 may also remain in a neutral position for a range of irradiation angles, thus providing a treatment chamber floor that can be walked on and driven on even while the patient 12 is being treated.

In addition, due to the disposition of the imaging device 50 on the movable floor 22, the imaging device 50 in the treatment chamber 6 may be used to obtain images during radiation treatment. As the floor segment 22 always remains in the treatment chamber 6 regardless of the position of the gantry 4, the imaging device 50 may be conveniently positioned for imaging. The weight of the imaging device 50 may be supported by the floor segment 22 and the gantry 4.

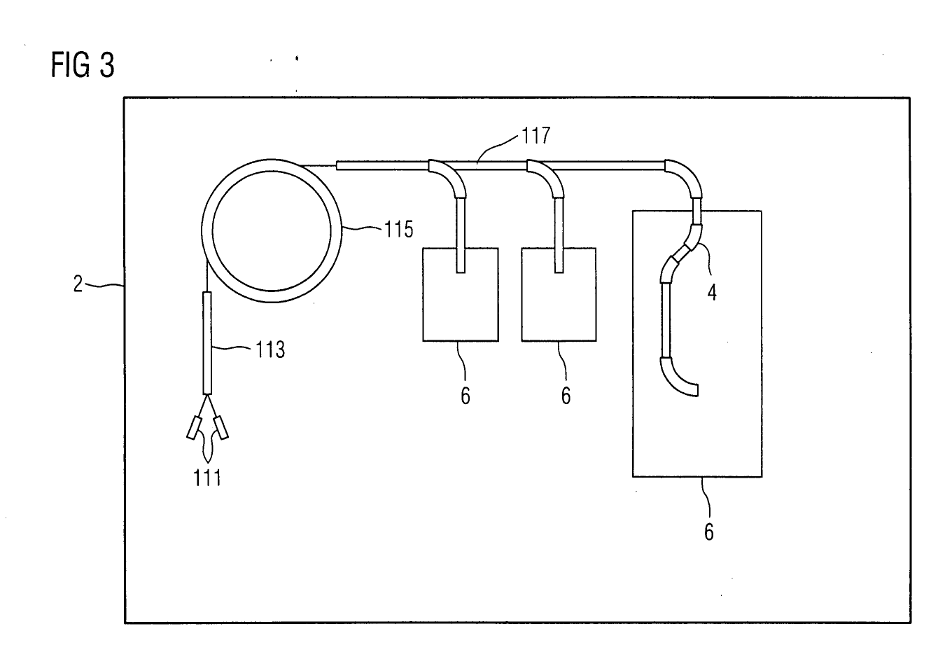

FIG. 3 shows a schematic representation of the construction of a particle therapy system 2. In a particle therapy system 2, the irradiation of a patient is done with a particle beam. Ions, such as protons, pions, helium ions, carbon ions, or other kinds of ions are the particles primarily used.

Such particles may be generated in a particle source 111 and accelerated in a preaccelerator 113, such as a linear accelerator (LINAC). The particles may then be fed into an accelerator 115, such as a synchrotron or cyclotron, in which they are accelerated to the energies needed for the irradiation. Once the particles leave the accelerator 115, a high-energy-beam transport system 117 guides the particle beam to the desired treatment chambers 6. In a treatment chamber 6, the accelerated particles are aimed at a patient. Depending on the configuration, this may be done from a fixed direction (in so-called “fixed-beam” chambers), or from different directions using a movable, rotatable gantry 4. In these last treatment chambers 6, the disposition of the imaging device on a movable floor is employed, of the kind described for instance above in conjunction with FIGS. 1 and 2. The construction of the particle therapy system 2 is representative of such systems, however a variety of configurations are known for the production of and guidance of the particle beam.

This fundamental construction of a particle therapy system 2 is typical for many particle therapy systems but can also deviate from them.

Although only a few exemplary embodiments of this invention have been described in detail above, those skilled in the art will readily appreciate that many modifications are possible in the exemplary embodiments without materially departing from the novel teachings and advantages of the invention. Accordingly, all such modifications are intended to be included within the scope of this invention as defined in the following claims.

What is claimed is:

1. A particle therapy system, including
a treatment chamber;
a rotatable gantry with a radiation treatment unit, capable of emitting a particle beam;
a movably supported floor disposed in the treatment chamber; and.
an imaging device disposed on the movably supported floor,
wherein the movably supported floor bears the weight of the imaging device.

2. The system of claim 1, wherein the movably supported floor is rotatable about a same axis as the rotatable gantry.

3. The system of claim 1, wherein the movably supported floor has at least one floor segment on which the imaging device is disposed

4. The system of claim 3, wherein the movably supported floor segment remains inside the treatment chamber for angular rotations of the gantry of plus or minus about 90 degrees with respect to a neutral position.

5. The system of claim 4, wherein the movably supported floor segment remains inside the treatment chamber for angular rotations of the gantry of plus or minus about 180 degrees with respect to a neutral position.

6. The system of claim 1, wherein the movably supported floor is a one-piece floor segment.

7. The system of claim 1, wherein at least a portion of the movably supported floor on which the imaging apparatus is disposed is surrounded by the gantry.

8. The system of claim 1, wherein the movably supported floor is movably supported on the gantry.

9. The system of claim 8, wherein the movably supported floor is supported with respect to the gantry by roller bearings.

10. The system of claim 8, wherein the movably supported floor is rotated by contact with the gantry.

11. The system of claim 1, wherein at least one of the movably supported floor or the gantry is provided with a resilient contact surface where a contact between the movably supported floor and the gantry is possible.

12. The system of claim 1, wherein the imaging device further comprises
a mounting device and an imaging unit, and the imaging unit is positionable by the mounting device.

13. The system of claim 12, wherein the mounting device is a robotic arm having a plurality of joints.

14. The system a claim 13, wherein the imaging unit is positionable in a parked position proximal to a back wall of the gantry.

15. The system of claim 14, wherein a patient support device is introducible through an opening in the gantry disposed on an opposite end of the gantry from the back wall.

16. The system of claim 12, wherein the imaging unit is a C-arm X-ray device.

17. The system of claim 7, wherein the movably supported floor is movably supported on the gantry.

18. The system of claim 17, wherein at least one of the movably supported floor or the gantry is provided with a resilient contact surface where a contact between the movably supported floor and the gantry is possible.

19. The system of claim 18, wherein the imaging device further comprises:
a mounting device and an imaging unit, and the imaging unit is positionable by the mounting device.
